(19) United States
(12) Patent Application Publication
(10) Pub. No.: US 2004/0205905 A1
(43) Pub. Date: Oct. 21, 2004

(54) Dye composition for keratin fibers, comprising at least one compound chosen from ortho- and alpha-dialdehyde compounds and at least one sulphur compound

(76) Inventor: Gregory Plos, Tokyo (JP)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/735,748

(22) Filed: Dec. 16, 2003

Related U.S. Application Data
(60) Provisional application No. 60/456,180, filed on Mar. 21, 2003.

(30) Foreign Application Priority Data
Dec. 16, 2002 (FR) 02 15913

(51) Int. Cl. A61K 7/13

(52) U.S. Cl. 8/405

(57) ABSTRACT

Disclosed herein is a dye composition for keratin fibers, for example, human keratin fibers such as the hair, comprising, in a medium suitable for dyeing, at least one compound chosen from ortho- and α-dialdehyde compounds as defined herein and at least one sulphur compound as defined herein. Also disclosed is the use of this composition for dyeing and/or optically lightening keratin fibers, and also to the dyeing process using this composition.

This application claims benefit of U.S. Provisional Application No. 60/456,180, filed Mar. 21, 2003.

Disclosed herein is a dye composition for keratin fibers, for example human keratin fibers such as the hair, comprising, in a medium suitable for dyeing, at least one compound chosen from ortho- and α-dialdehyde compounds of formula (I) defined herein and at least one sulphur compound chosen from compounds of formula (II) and (III) as defined herein.

Conventionally, the lightening of keratin fibers may be obtained by using oxidizing agents such as persalts or aqueous hydrogen peroxide solution, which may degrade the natural melanin pigments and/or artificial pigments present in the hair. This process of chemical lightening is typically efficient in terms of lightening, but may have the drawback of being aggressive to the hair.

Accordingly, disclosed herein are novel compositions for lightening keratin fibers, which lack at least one of the drawbacks of the compositions of the prior art. In other words, disclosed herein is a novel dyeing system which may simultaneously have at least one of: the advantage of fastness, such as with respect to repeated shampooing, and the advantage of friendliness towards hair fibers.

The novel composition disclosed herein is a dye composition for keratin fibers, comprising, in a medium that is suitable for dyeing:

at least one compound chosen from ortho- and α-dialdehyde compounds of formula (I):

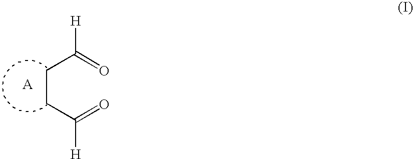

wherein A is chosen from:

fused and non-fused, aromatic and non-aromatic monocarbocyclic and polycarbocyclic groups comprising from 6 to 50 carbon atoms;

fused and non-fused, aromatic and non-aromatic, 5- to 30-membered monoheterocyclic and polyheterocyclic groups comprising at least one hetero atom chosen from nitrogen, sulphur, oxygen and phosphorus;

the group A possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, nitro, sulphonato, ammonio, C₁-C₄ trialkylammonio, imidazolio, pyridinio and benzothiazolio radicals; and

at least one sulphur compound chosen from compounds of formula (II) and (III):

R—SH (II)

R—S—S—R′ (III)

wherein R and R′, which may be identical or different, are chosen from saturated and unsaturated, branched and unbranched groups comprising from 1 to 100 carbon atoms, optionally comprising from 1 to 30 unsaturations, and which may comprise at least one hetero atom chosen from nitrogen, sulphur, oxygen and phosphorus, R and R′ possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, amino, C₁-C₄ monoalkylamino, C₁-C₄ dialkylamino, C₁-C₄ monohydroxyalkylamino, C₁-C₄ dihydroxyalkylamino, nitro, sulphonato, ammonio, C₁-C₄ trialkylammonio, imidazolio, pyridinio and benzothiazolio radicals.

With the composition disclosed herein, a fluorescent coloration may be obtained while at the same time interacting with the amino acids present in the hair. This may result in great fastness of the coloration obtained over time, even if the hair is subjected to repeated washing.

With the composition disclosed herein, keratin fibers may also be colored without being degraded, since the composition may be used in the absence of oxidizing agents. With this composition, the dyeing result obtained may make it possible to obtain optical lightening of the fiber.

Also disclosed herein is a process for dyeing keratin fibers, for example human keratin fibers such as the hair, using the disclosed composition, and also a multi-compartment device for performing this process.

In addition, disclosed herein is the use of the composition for dyeing and/or optically lightening keratin fibers, for example human keratin fiber such as the hair.

As used herein, the term “optical lightening” means a visual effect of lightening naturally or artificially colored keratin fibers, without using compounds that destroy the natural or artificial colored pigments present in the keratin fibers.

As used herein, the term “fused” means at least two conjoined rings containing at least two atoms in common.

The at least one group chosen from fused and non-fused, aromatic and non-aromatic monocarbocyclic and polycarbocyclic groups comprising from 6 to 50 carbon atoms as used herein may be chosen from, for example, benzene, naphthalene, and anthracene rings.

The at least one group chosen from fused and non-fused, aromatic and non-aromatic, 5- to 30-membered monoheterocyclic and polyheterocyclic groups comprising at least one hetero atom may be chosen from, for example, thiophene, benzofuran, benzothiophene, indole, bispyridine, benzopyran, quinoline, pyrazole, pyridine, pyrrole, furan, imidazole, and benzimidazole ring systems. The polyheterocyclic group may be fused or substituted with at least one carbocyclic group.

As used herein, the term “alkyl radical” (alk) means a radical chosen from linear and branched alkyl radicals, for example methyl, ethyl, n-propyl, isopropyl and butyl. Also as used herein, an alkoxy radical is a radical alk-O—, an alkylthio radical is a radical alk-S—, an alkylcarbonyl radical is a radical alk-CO—, an alkylcarboxyl radical is a radical alk-COO—, a monoalkylamino or dialkylamino radical is a radical —N(alk)ₙ with n=1 or 2, a monohydroxyalkylamino or dihydroxyalkylamino radical is a radical —N(alk-OH)ₘ with m=1 or 2, wherein in each of these definitions the alkyl radical has the definition given above.

As used herein, a sulphonato radical is a radical —SO₃⁻. Further as used herein, a trialkylammonio radical is a radical (alk)₃N⁺— with the alkyl radical having the definition given above, and the imidazolio, pyridinio and benzothiazolio radicals are cationic radicals corresponding to the imidazolium, pyridinium and benzothiazolium cations.

As used in the present disclosure, a “halo group” denotes a halogen atom chosen from, for example, chlorine, bromine and iodine.

The term “unsaturations” as used herein means double or triple bonds and these bonds may lead to aromatic or heteroaromatic structures.

The term “amino acid” as used herein means any organic structure containing a carboxylic acid end function and an amine end function.

In one embodiment of the composition disclosed herein, the at least one compound chosen from ortho- and α-dialdehyde compounds of formula (I) is chosen from ortho-phthalaldehyde derivatives of formula (IV):

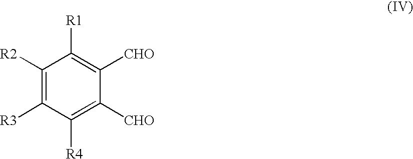

wherein R₁, R₂, R₃ and R₄ are each independently chosen from a hydrogen atom, from halo, C₁-C₄ alkyl, hydroxyl, C₁-C₄ alkoxy, C₁-C₄ alkylthio, C₁-C₄ alkylcarboxyl, nitro, and sulphonato radicals, and from 5- to 8-membered non-aromatic heterocyclic nitrogenous groups.

The groups R₁, R₂, R₃ and R₄ may, for example, be chosen from C₁-C₄ alkyl and C₁-C₄ alkoxy groups.

For example, the ortho-phthaladehyde derivatives of formula (IV) may be chosen from ortho-phthaladehyde and 4,5-dimethoxyphthaladehyde.

As another example of the composition disclosed herein, the at least one compound chosen from ortho- and α-dialdehyde compounds of formula (I) may be chosen from naphthalenedicarboxaldehyde, anthracenedicarboxaldehyde and thiophenedicarboxaldehyde derivatives.

In another embodiment, the at least one compound chosen from ortho- and α-dialdehyde compounds of formula (I) may be chosen from 1,2-naphthalenedicarboxaldehyde, 2,3-naphthalenedicarboxaldehyde, 2,3-anthracenedicarboxaldehyde and 2,3-thiophenedicarboxaldehyde.

With respect to the at least one sulphur compound chosen from compounds of formula (II) and formula (III), R and R′ in formulas (II) and (III) may comprise, for example, from 1 to 4 carbon atoms.

The sulphur compound of formula (II), for example, may be chosen from ethanethiol, 2-aminothiophenol, thioglycolic acid and cysteamine. The sulphur compound of formula (III), for example, may be cystamine.

The composition disclosed herein may also contain at least one amino acid chosen from natural amino acids and synthetic amino acids of D form or of L form.

By way of example, the at least one amino acid may be chosen from glycine, alanine, valine, leucine, isoleucine, methionine, phenylalanine, proline, serine, threonine, cysteine, asparagine, glutamine, tyrosine, histidine, lysine, ornithine, arginine, aspartic acid, glutamic acid and tryptophan.

The composition disclosed herein may also contain at least one peptide and/or at least one protein.

The concentrations of the various components of the composition disclosed herein depend on the strength of the coloration that it is desired to obtain. However, the concentration of the at least one compound chosen from ortho- and α-dialdehyde compounds of formula (I) may range from 0.01% to 30% by weight, such as from 0.05% to 20% by weight, relative to the total weight of the composition; the concentration of the at least one sulphur compound chosen from compounds of formula (II) and (III) may range from 0.01% to 30% by weight, such as from 0.05% to 20% by weight, relative to the total weight of the composition; and the concentration of the at least one amino acid, when they are present, ranges from 0.01% to 20% by weight, such as from 0.05% to 30% by weight relative to the total weight of the composition.

The composition disclosed herein may also comprise at least one compound chosen from surfactants, polyols, polyol ethers, and aromatic monoalcohols. The individual concentration of surfactants, polyols, polyol ethers, and aromatic monoalcohols, when they are present, may range from 0.1% to 20% by weight, such as from 0.5% to 30% by weight, relative to the total weight of the composition.

The surfactant(s) may be chosen, without preference, alone or as mixtures, from anionic, amphoteric, nonionic, zwitterionic, and cationic surfactants.

As examples of anionic surfactants that can be used, alone or as mixtures, in the context of the composition disclosed herein, non-limiting mention may be made, of salts, such as alkaline salts, for example sodium salts, ammonium salts, amine salts, amino alcohol salts and magnesium salts of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄) alkyl sulphosuccinates, (C₆-C₂₄) alkyl ether sulphosuccinates, (C₆-C₂₄) alkylamide sulphosuccinates; (C₆-C₂₄) alkyl sulphoacetates; (C₆-C₂₄) acyl sarcosinates and (C₆-C₂₄) acyl glutamates. It is also possible to use the carboxylic esters of (C₆-C₂₄) alkylpolyglycosides, such as alkylglucoside citrates, alkylpolyglycoside tartrates and alkylpolyglycoside sulphosuccinates, alkylsulphosuccinamates; acyl isethionates and N-acyltaurates, wherein the alkyl or acyl radical of all of these various compounds may comprise, for example, from 12 to 20 carbon atoms, and the aryl radical, for example, may be chosen from phenyl and benzyl groups. Among the anionic surfactants which can also be used, non-limiting mention may also be made of fatty acid salts such as the salts of oleic, ricinoleic, palmitic, and stearic acids, coconut oil acid and hydrogenated coconut oil acid; acyl lactylates in which the acyl radical contains 8 to 20 carbon atoms. Alkyl-D-galactosideuronic acids and their salts, polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, such as those containing from 2 to 50 alkylene oxide, for example, ethylene oxide, groups, and mixtures thereof can also be used.

The nonionic surfactants useful herein are also compounds that are well known per se (see for instance “Handbook of Surfactants” by M. R. Porter, published by Blackie & Son (Glasgow and London), 1991, pp. 116-178) and, in the present context, their nature is not a critical feature. Thus, they can be chosen, for example, from polyethoxylated alkylphenols and polypropoxylated alkylphenols, α-diols, and alcohols having a fatty chain containing, for example, 8 to 18 carbon atoms, it being possible for the number of ethylene oxide or propylene oxide groups to range, for example, from 2 to 50. Non-limiting mention may also be made of copolymers of ethylene oxide and of propylene oxide, condensates of ethylene oxide and of propylene oxide with fatty alcohols; polyethoxylated fatty amides having, for example, from 2 to 30 mol of ethylene oxide, polyglycerolated fatty amides containing on average 1 to 5, such as 1.5 to 4, glycerol groups; polyethoxylated fatty amines having, for example, from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. In one embodiment, alkylpolyglycosides may be used as the nonionic surfactant(s), as well as the ethoxylated fatty alcohols.

The amphoteric and zwitterionic surfactants, whose nature is not a critical feature herein, can be, for example, chosen from aliphatic secondary and tertiary amine derivatives in which the aliphatic radical is chosen from linear and branched chains containing 8 to 18 carbon atoms and containing at least one water-soluble anionic group, for example carboxylate, sulphonate, sulphate, phosphate, and phosphonate; non-limiting mention may also be made of (C₈-C₂₀)alkylbetaines, sulphobetaines, (C₈-C₂₀)alkylamido-(C₁-C₆)alkylbetaines and (C₈-C₂₀)alkylamido(C₁-C₆)alkylsulphobetaines.

Among the amine derivatives useful as amphoteric or zwitterionic surfactants, non-limiting mention may be made of the products sold under the name MIRANOL, as described in U.S. Pat. Nos. 2,528,378 and 2,781,354 and classified in the CTFA dictionary, 3rd edition, 1982, under the names “amphocarboxyglycinates” and “amphocarboxypropionates” of respective structures:

R₃₄—CONHCH₂CH₂—N(R₃₅)(R₃₆)(CH₂COO⁻)

in which:

R₃₄ is chosen from an alkyl radical of an acid R₃₄—COOH present in hydrolyzed coconut oil, and from alkyl radicals chosen from heptyl, nonyl, and undecyl radicals;

R₃₅ is a β-hydroxyethyl group and

R₃₆ is a carboxymethyl group; and

R_{34′}—CONHCH₂CH₂—N(B)(D)

in which:

B is —CH₂CH₂OX′, D is —(CH₂)_{z}—Y′, with z=1 or 2,

X′ is chosen from a —CH₂CH₂—COOH group and a hydrogen atom,

Y′ is chosen from —COOH and a —CH₂—CHOH—SO₃H radical,

R₃₄′ is chosen from an alkyl radical of an acid R₃₇—COOH present in coconut oil and in hydrolyzed linseed oil; an alkyl radical, such as a C₇, C₉, C₁₁, and C₁₃ alkyl radical; a C₁₇ alkyl radical and its iso form; and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

For example, non-limiting mention may be made of the cocoamphodiacetate sold under the trade name Miranol® C2M Concentrate by the company Rhodia Chimie.

Among the cationic surfactants that may be used herein, non-limiting mention may be made of: primary, secondary and tertiary fatty amine salts, optionally polyoxyalkylenated; quaternary ammonium salts such as tetraalkylammonium, alkylamidoalkyltrialkylammonium, trialkylbenzylammonium, trialkylhydroxyalkylammonium and alkylpyridinium chlorides and bromides; imidazoline derivatives; and amine oxides of cationic nature.

The polyols disclosed herein are compounds containing from 2 to 100, such as from 3 to 50, carbon atoms, and from 2 to 25, such as from 2 to 10, hydroxyl radicals. Their molecular weight may be, for example, less than 500. Among the polyols that may be used herein, non-limiting mention may be made of propylene glycol, glycerol, hexylene glycol, butylene glycol, isopropene glycol, neopentyl glycol, and polyethylene glycols.

Among the polyol ethers that may be used herein, non-limiting mention may be made of propylene glycol monomethyl ether and dipropylene glycol monomethyl ether.

The aromatic monoalcohols that may be used contain from 6 to 50 carbon atoms. The aromatic ring system may be monocyclic or polycyclic. One aromatic monoalcohol that may be used, for example, is benzyl alcohol.

The composition disclosed herein may also contain at least one direct dye, which may be chosen, for instance, from nitrobenzene dyes, azo direct dyes and methine direct dyes. These direct dyes may be chosen from those of nonanionic, anionic, and cationic nature.

The composition disclosed herein may also comprise at least one oxidation base conventionally used in oxidation dyeing. By way of example, these oxidation bases may be chosen from para-phenylenediamines, bis(phenyl)alkylenediamines, para-aminophenols, ortho-aminophenols, and heterocyclic bases, and the addition salts thereof.

The present composition may also comprise at least one coupler conventionally used in oxidation dyeing. By way of example, these couplers may be chosen from meta-phenylenediamines, meta-aminophenols, meta-diphenols, naphthalene-based couplers, and heterocyclic couplers, and the addition salts thereof.

The medium that is suitable for dyeing, also known as the dye support, comprises water or a mixture of water and of at least one organic solvent to dissolve the compounds that would not be sufficiently water-soluble. Non-limiting examples of organic solvents that may be mentioned include C₁-C₄ lower alkanols, such as ethanol and isopropanol.

The solvents may, for example, be present in proportions ranging from 1% to 40% by weight approximately, relative to the total weight of the dye composition, for example, from 5% to 30% by weight approximately.

The composition disclosed herein may also contain at least one of various adjuvants conventionally used in hair dye compositions, such as anionic, cationic, nonionic, amphoteric, and zwitterionic polymers, and mixtures thereof, mineral and organic thickeners, such as anionic, cationic, nonionic and amphoteric polymeric associative thickeners, antioxidants, penetrating agents, sequestrants, fragrances, buffers, dispersants, conditioners, for example volatile and non-volatile, modified and unmodified silicones, film-forming agents, ceramides, preserving agents, and opacifiers.

The above adjuvants may be present in an amount for each adjuvant ranging from 0.01% to 20% by weight relative to the total weight of the composition.

Needless to say, a person skilled in the art will take care to select this or these optional additional compound(s) such that at least one advantageous property intrinsically associated with the optical lightening composition in accordance with the invention is not, or is not substantially, adversely affected by the envisaged addition(s).

The pH of the present composition ranges, for example, from 4 to 12, for further example, from 5 to 11. It may be adjusted to the desired value using acidifying or basifying agents usually used in the dyeing of keratin fibers, or alternatively using standard buffer systems.

Among the acidifying agents that may be mentioned, for example, are mineral and organic acids, for instance hydrochloric acid, orthophosphoric acid, sulphuric acid, carboxylic acids, for instance acetic acid, tartaric acid, citric acid, and lactic acid, and sulphonic acids.

Among the basifying agents that may be mentioned, for example, are aqueous ammonia, alkaline carbonates, alkanolamines, such as monoethanolamine, diethanolamine, and triethanolamine, and also derivatives thereof, sodium hydroxide, potassium hydroxide and the compounds of formula (V) below:

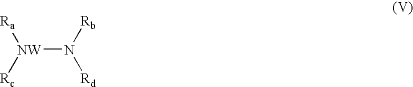

in which W is a propylene residue optionally substituted with a substituent chosen from hydroxyl group and C₁-C₄ alkyl radicals; Rₐ, R_{b}, R_{c} and R_{d}, which may be identical or different, are chosen from a hydrogen atom, C₁-C₄ alkyl radicals, and C₁-C₄ hydroxyalkyl radicals.

The composition disclosed herein may be in various forms, such as in the form of liquids, creams, and gels, or in any other form that is suitable for dyeing keratin fibers, and especially human hair.

The process for dyeing keratin fibers of the present invention comprises applying a composition (a) comprising, in a medium suitable for dyeing, at least one compound chosen from ortho- and α-dialdehyde compounds of formula (I) and a composition (b) comprising, in a medium suitable for dyeing, at least one sulphur compound chosen from compounds of formulas (II) and (III) to keratin fibers for a leave-in time that is sufficient to obtain the desired coloration.

According to one embodiment, composition (b) furthermore may comprise at least one amino acid.

In another embodiment, a composition (c) comprising, in a medium suitable for dyeing, at least one amino acid is also applied to the keratin fibers.

In another embodiment, compositions (a), (b), and/or (c) may also comprise a compound chosen from surfactants, polyols, polyol ethers, and aromatic monoalcohols.

In a first variant of this process, compositions (a), (b), and optionally (c) are mixed together just before use, and the mixture thus obtained is applied to the keratin fibers for a leave-in time that is sufficient to obtain the desired coloration.

In a second variant of this process, compositions (a), (b), and optionally (c) are applied successively to the keratin fibers for a leave-in time that is sufficient to obtain the desired coloration, the various compositions being applied in any order.

The leave-in time ranges, for example, from 5 minutes to 1 hour, for further example, from 5 minutes to 30 minutes.

The application temperature ranges from room temperature to 80° C, for example, from room temperature to 60° C.

Also disclosed herein is a multi-compartment device for performing the process for dyeing keratin fibers described above.

The multi-compartment device comprises a first compartment comprising a composition (a) comprising, in a medium suitable for dyeing, at least one compound chosen from ortho- and α-dialdehyde compounds of formula (I) and a second compartment comprising a composition (b) comprising, in a medium suitable for dyeing, at least one sulphur compound chosen from compounds of formula (II) and of formula (III).

In one embodiment of the disclosed device, composition (b) also comprises at least one amino acid.

In another embodiment of the disclosed device, the device also comprises a third compartment comprising a composition (c) comprising, in a medium suitable for dyeing, at least one amino acid.

In another embodiment of the device, compositions (a), (b), and/or (c) also comprise a compound chosen from surfactants, polyols, polyol ethers, and aromatic monoalcohols.

This device may be equipped with a means for applying the desired mixture to the hair, such as the devices described in patent FR-2 586 913.

In addition, disclosed herein is also the use, for dyeing and/or optically lightening keratin fibers, of a composition comprising at least one compound chosen from ortho- and α-dialdehyde compounds of formula (I) and at least one sulphur compound chosen from compounds of formula (II) and formula (III).

In one embodiment, the composition used may also comprise at least one amino acid.

In another embodiment, the composition used may also comprise a compound chosen from surfactants, polyols, polyol ethers, and aromatic monoalcohols.

The examples that follow serve to illustrate the invention without, however, being limiting in nature.

### EXAMPLES

In the examples that follow, the amounts of the compounds are expressed in grams per 100 g of composition (%) or in moles per 100 g of composition (mol %).

### Example 1

Composition 1 was prepared as below:

| Compounds | Amount |
|---|---|
| ortho-Phthalaldehyde | 0.5% |
| Cysteamine dihydrochloride | 3 × 10⁻³ mol % |
| Glycine | 10⁻³ mol % |
| NaOH | qs pH 11 |
| Distilled water | qs 100 g |

This composition was applied to locks of natural or permanent-waved grey hair containing 90% white hairs. The ratio of the amount of composition to the amount of hair was equal to 5, and the application temperature was equal to room temperature. After a leave-in time of 30 minutes, the locks were rinsed, shampooed, and dried.

The coloration obtained was measured using a Minolta CM2002 spectrocolorimeter (specular components excluded, illuminant D65, angle 10°). The calorimetric results are given in the table below:

| | L* | a* | b* | Color |
|---|---|---|---|---|
| Composition 1 - natural hair | 45.40 | 0.70 | 22.55 | Yellow |
| Composition 1 - permanent-waved | 41.35 | 1.20 | 24.80 | Yellow |
| hair | | | | |

This composition applied to chestnut-brown hair produced a very attractive lightening optical effect.

### Example 2

Composition 2 is prepared as below:

| Compounds | Amount |
|---|---|
| Ortho-Phthalaldehyde | 0.4% |
| Cysteamine dihydrochloride | 3.8% |
| Distilled water | qs 100 g |

This composition was applied to locks of natural or permanent-waved grey hair containing 90% white hairs. The ratio of the amount of composition to the amount of hair was equal to 5, and the application temperature was equal to room temperature. After a leave-in time of 15 minutes, the locks were rinsed, shampooed, and dried.

The coloration obtained was measured using a Minolta CM2002 spectrocolorimeter (specular components excluded, illuminant D65, angle 10°). The calorimetric results are given in the table below:

| | L* | a* | b* | Color |
|---|---|---|---|---|
| Composition 1 - natural hair | 47.00 | 4.15 | 20.80 | Yellow |
| Composition 1 - permanent-waved | 46.35 | 4.10 | 22.70 | Yellow |
| hair | | | | |

In the present case, there was interaction of the composition with the amino acids present in the hair, thus making it possible to obtain fast optical lightening on chestnut-brown hair, i.e. optical lightening that withstood shampooing and did not degrade the keratin fibers.

### Examples 3 to 7

Compositions 3 to 7 were prepared as below:

| | Compositions | | | | |
|---|---|---|---|---|---|
| | 3 | 4 | 5 | 6 | 7 |
| ortho-Phthalaldehyde | 0.5% | 0.5% | 0.4% | 0.4% | 0.4% |
| Cysteamine | 0.034% | 0.034% | 0.013% | 0.013% | 0.013% |
| Glycine | 0.0075% | 0.0075% | — | — | — |
| Benzyl alcohol | 1% | — | 1% | — | — |
| Sodium lauryl ether | — | 1% | — | 1% | — |
| sulphate (A.M.) | | | | | |
| NaOH | qs pH 11 | qs pH 11 | — | — | — |
| Distilled water | qs 100% | qs 100% | qs 100% | qs 100% | qs 100% |

These compositions were applied to locks of natural or permanent-waved grey hair containing 90% white hairs. The ratio of the amount of composition to the amount of hair was equal to 5, and the application temperature was equal to room temperature. After a leave-in time of 30 minutes, the locks were rinsed, shampooed, and dried.

The colorations obtained were measured using a Minolta CM3600d spectrocolorimeter (specular components included, illuminant D65, angle 10°). The calorimetric results are given in the table below:

| | L* | a* | b* |
|---|---|---|---|
| Composition 3 - natural hair | 37.75 | 6.50 | 7.40 |
| Composition 3 - permanent-waved hair | 27.30 | 7.85 | 5.35 |
| Composition 4 - natural hair | 40.30 | 5.30 | 6.30 |
| Composition 4 - permanent-waved hair | 29.95 | 8.55 | 6.55 |
| Composition 5 - natural hair | 40.35 | 2.10 | 17.15 |
| Composition 5 - permanent-waved hair | 39.00 | 4.65 | 17.65 |
| Composition 6 - natural hair | 51.70 | −1.70 | 13.30 |
| Composition 6 - permanent-waved hair | 41.95 | 1.70 | 16.80 |
| Composition 7 - natural hair | 44.80 | 2.95 | 20.65 |
| Composition 7 - permanent-waved hair | 40.95 | 4.00 | 17.60 |

### Examples 8 to 12

Compositions 8 to 12 were prepared as below:

| | Compositions | | | | |
|---|---|---|---|---|---|
| | 8 | 9 | 10 | 11 | 12 |
| ortho-Phthalaldehyde | 0.5% | 0.5% | 0.4% | 0.4% | 0.4% |
| Cysteamine | 3 × 10⁻³ mol % | 3 × 10⁻³ mol % | 3 × 10⁻³ mol % | 3 × 10⁻³ mol % | 3 × 10⁻³ mol % |
| Glycine | 10⁻³ mol % | 10⁻³ mol % | 10⁻³ mol % | — | — |
| Benzyl alcohol | 1% | — | 1% | — | — |
| Sodium lauryl ether | — | 1% | — | 1% | — |
| sulphate (A.M.) | | | | | |
| NaOH | qs pH 11 | qs pH 11 | — | — | — |
| Distilled water | qs 100% | qs 100% | qs 100% | qs 100% | qs 100% |

These compositions were applied to locks of natural or permanent-waved grey hair containing 90% white hairs. The ratio of the amount of composition to the amount of hair was equal to 5, and the application temperature was equal to room temperature. After a leave-in time of 30 minutes, the locks were rinsed, shampooed, and dried.

The colorations obtained were measured using a Minolta CM3600d spectrocolorimeter (specular components included, illuminant D65, angle 10°). The calorimetric results are given in the table below:

| | L* | a* | b* |
|---|---|---|---|
| Composition 8 - natural hair | 49.85 | 2.55 | 16.70 |
| Composition 8 - permanent-waved hair | 50.00 | 4.05 | 20.45 |
| Composition 9 - natural hair | 57.00 | 0.90 | 15.35 |
| Composition 9 - permanent-waved hair | 55.00 | 3.65 | 26.25 |
| Composition 10 - natural hair | 50.25 | 6.40 | 36.90 |
| Composition 10 - permanent-waved hair | 52.90 | 2.05 | 22.80 |
| Composition 11 - natural hair | 43.65 | 6.30 | 22.10 |
| Composition 11 - permanent-waved hair | 49.20 | 2.25 | 15.30 |
| Composition 12 - natural hair | 51.70 | 4.30 | 29.35 |
| Composition 12 - permanent-waved hair | 51.00 | 3.50 | 28.35 |

What is claimed is:

1. A composition for the dyeing of keratin fibers, comprising, in a medium suitable for dyeing:
at least one compound chosen from ortho- and α-dialdehyde compounds of formula (I): 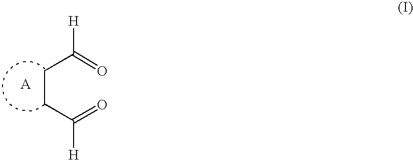
wherein A is chosen from:
fused and non-fused, aromatic and non-aromatic monocarbocyclic and polycarbocyclic groups comprising from 6 to 50 carbon atoms;
fused and non-fused, aromatic and non-aromatic, 5- to 30-membered monoheterocyclic and polyheterocyclic groups comprising at least one hetero atom chosen from nitrogen, sulphur, oxygen and phosphorus;
the group A possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, nitro, sulphonato, ammonio, C₁-C₄ trialkylammonio, imidazolio, pyridinio and benzothiazolio radicals;
at least one sulphur compound chosen from compounds of formula (II) and of formula (III):
R—SH (II)
R—S—S—R′ (III)
wherein R and R′, which may be identical or different, are chosen from saturated and unsaturated, branched and unbranched groups comprising from 1 to 100 carbon atoms, optionally comprising from 1 to 30 unsaturations, and which may comprise at least one hetero atom chosen from nitrogen, sulphur, oxygen and phosphorus, R and R′ possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, amino, C₁-C₄ monoalkylamino, C₁-C₄ dialkylamino, C₁-C₄ monohydroxyalkylamino, C₁-C₄ dihydroxyalkylamino, nitro, sulphonato, ammonio, C₁-C₄ trialkylammonio, imidazolio, pyridinio and benzothiazolio radicals.

2. The composition according to claim 1, wherein the at least one compound chosen from ortho- and α-dialdehyde compounds of formula (I) is chosen from ortho-phthalaldehyde derivatives of formula (IV): 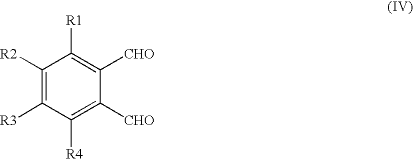
wherein R₁, R₂, R₃ and R₄ are each independently chosen from a hydrogen atom, from halo, C₁-C₄ alkyl, hydroxyl, C₁-C₄ alkoxy, C₁-C₄ alkylthio, C₁-C₄ alkylcarboxyl, nitro and sulphonato radicals, and from 5- to 8-membered non-aromatic heterocyclic nitrogenous groups.

3. The composition according to claim 2, wherein the groups R₁, R₂, R₃ and R₄ are each independently chosen from C₁-C₄ alkyl and C₁-C₄ alkoxy groups.

4. The composition according to claim 3, wherein the ortho-phthalaldehyde derivatives of formula (IV) are chosen from ortho-phthalaldehyde and 4,5-dimethoxyphthalaldehyde.

5. The composition according to claim 1, wherein the at least one compound chosen from ortho- and α-dialdehyde compounds of formula (I) is chosen from naphthalenedicarboxaldehyde, anthracenedicarboxaldehyde and thiophenedicarboxaldehyde derivatives.

6. The composition according to claim 5, wherein the at least one compound chosen from ortho- and α-dialdehyde compounds of formula (I) is chosen from 1,2-naphthalenedicarboxaldehyde, 2,3-napthalenedicarboxaldehyde, 2,3-anthracenedicarboxaldehyde and 2,3-thiophenedicarboxaldehyde.

7. The composition according to claim 1, in which R and R′ in formulas (II) and (III), which may be identical or different, each contains from 1 to 4 carbon atoms.

8. The composition according to claim 7, wherein the at least one sulphur compound chosen from compounds of formula (II) and formula (III) is chosen from ethanethiol, 2-aminothiophenol, thioglycolic acid, cysteamine and cystamine.

9. The composition according to claim 1, wherein the at least one compound chosen from ortho- and α-dialdehyde compounds of formula (I) is present in the composition in an amount ranging from 0.01% to 30% by weight relative to the total weight of the composition.

10. The composition according to claim 1, wherein the at least one sulphur compound chosen from compounds of formula (II) and formula (III) is present in the composition in an amount ranging from 0.01% to 30% by weight relative to the total weight of the composition.

11. The composition according to claim 1, further comprising at least one amino acid.

12. The composition according to claim 11, wherein the at least one amino acid is chosen from glycine, alanine, valine, leucine, isoleucine, methionine, phenylalanine, proline, serine, threonine, cysteine, asparagine, glutamine, tyrosine, histidine, lysine, ornithine, arginine, aspartic acid, glutamic acid and tryptophan.

13. The composition according to claim 11, wherein the at least one amino acid is present in the composition in an amount ranging from 0.01% to 20% by weight relative to the total weight of the composition.

14. The composition according to claim 1, further comprising at least one peptide.

15. The composition according to claim 1, further comprising at least one protein.

16. The composition according to claim 1, further comprising at least one surfactant.

17. The composition according to claim 16, in which the at least one surfactant is chosen from anionic, amphoteric, nonionic, zwitterionic and cationic surfactants.

18. The composition according to claim 1, further comprising at least one compound chosen from polyols, polyol ethers, and aromatic monoalcohols.

19. The composition according to claim 18, wherein the polyols comprise from 2 to 100 carbon atoms and from 2 to 25 hydroxyl radicals, and have a molecular weight less than 500.

20. The composition according to claim 19, wherein the polyols are chosen from propylene glycol, glycerol, hexylene glycol, butylene glycol, isopropylene glycol, neopentyl glycol and polyethylene glycols.

21. The composition according to claim 18, wherein the polyol ethers are chosen from propylene glycol monomethyl ether and dipropylene glycol monomethyl ether.

22. The composition according to claim 18, wherein the aromatic monoalcohols are chosen from monocyclic and polycyclic compounds comprising from 6 to 50 carbon atoms.

23. The composition according to claim 22, wherein the aromatic monoalcohols are chosen from benzyl alcohol.

24. The composition according to claim 16, wherein the at least one surfactant is present in the composition in an amount ranging from 0.1% to 20% by weight relative to the total weight of the composition.

25. The composition according to claim 18, wherein the at least one compound chosen from polyols, polyol ethers, and aromatic monoalcohols is present in the composition in an amount ranging from 0.1% to 20% by weight relative to the total weight of the composition.

26. A process for dyeing keratin fibers, comprising applying to keratin fibers for a leave-in time sufficient to obtain a desired coloration:
a composition (a) comprising, in a medium suitable for dyeing, at least one compound chosen from ortho- and α-dialdehyde compounds of formula (I): 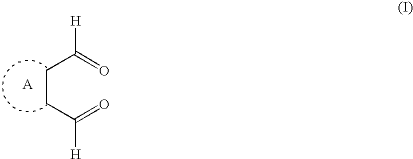
wherein A is chosen from:
fused and non-fused, aromatic and non-aromatic monocarbocyclic and polycarbocyclic groups comprising from 6 to 50 carbon atoms;
fused and non-fused, aromatic and non-aromatic, 5- to 30-membered monoheterocyclic and polyheterocyclic groups comprising at least one hetero atom chosen from nitrogen, sulphur, oxygen and phosphorus;
the group A possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, nitro, sulphonato, ammonio, C₁-C₄ trialkylammonio, imidazolio, pyridinio and benzothiazolio radicals; and
a composition (b) comprising at least one sulphur compound chosen from compounds of formula (II) and of formula (III):
R—SH (II)
R—S—S—R′ (III)
wherein R and R′, which may be identical or different, are chosen from saturated and unsaturated, branched and unbranched groups comprising from 1 to 100 carbon atoms, optionally comprising from 1 to 30 unsaturations, and which may comprise at least one hetero atom chosen from nitrogen, sulphur, oxygen and phosphorus, R and R′ possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, amino, C₁-C₄ monoalkylamino, C₁-C₄ dialkylamino, C₁-C₄ monohydroxyalkylamino, C₁-C₄ dihydroxyalkylamino, nitro, sulphonato, ammonio, C₁-C₄ trialkylammonio, imidazolio, pyridinio and benzothiazolio radicals.

27. The process according to claim 26, wherein composition (b) further comprises at least one amino acid.

28. The process according to claim 27, wherein the at least one amino acid is chosen from glycine, alanine, valine, leucine, isoleucine, methionine, phenylalanine, proline, serine, threonine, cysteine, asparagine, glutamine, tyrosine, histidine, lysine, ornithine, arginine, aspartic acid, glutamic acid and tryptophan.

29. The process according to claim 26, wherein at least one of compositions (a) and (b) further comprises at least one compound chosen from surfactants, polyols, polyol ethers, and aromatic monoalcohols.

30. The process according to claim 26, further comprising applying to the keratin fibers a composition (c) comprising, in a medium suitable for dyeing, at least one amino acid.

31. The process according to claim 30, wherein at least one of compositions (a), (b) and (c) furthers comprise at least one compound chosen from surfactants, polyols, polyol ethers, and aromatic monoalcohols.

32. The process according to claim 26, wherein compositions (a) and (b) are mixed together just before use and the mixture thus obtained is applied to the keratin fibers for a leave-in time sufficient to obtain the desired coloration.

33. The process according to claim 30, wherein compositions (a), (b) and (c) are mixed together just before use and the mixture thus obtained is applied to the keratin fibers for a leave-in time sufficient to obtain the desired coloration.

34. The process according to claim 26, wherein compositions (a) and (b) are applied successively to the keratin fibers with optional intermediate rinsing, for a leave-in time sufficient to obtain the desired coloration, said compositions (a) and (b) being applied in any order.

35. The process according to claim 30, in which compositions (a), (b) and (c) are applied, successively and optionally with intermediate rinsing, to the keratin fibers for a leave-in time sufficient to obtain the desired coloration, said compositions (a), (b) and (c) being applied in any order.

36. A multi-compartment device, comprising:
a first compartment comprising a composition (a) comprising, in a medium suitable for dyeing, at least one compound chosen from ortho- and α-dialdehyde compounds of formula (I): 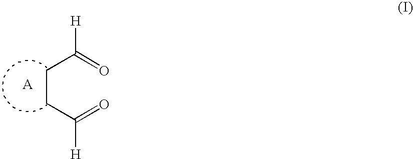
wherein A is chosen from:
fused and non-fused, aromatic and non-aromatic monocarbocyclic and polycarbocyclic groups comprising from 6 to 50 carbon atoms;
fused and non-fused, aromatic and non-aromatic, 5- to 30-membered monoheterocyclic and polyheterocyclic groups comprising at least one hetero atom chosen from nitrogen, sulphur, oxygen and phosphorus;
the group A possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, nitro, sulphonato, ammonio, C₁-C₄ trialkylammonio, imidazolio, pyridinio and benzothiazolio radicals; and
a second compartment comprising a composition (b) comprising, in a medium suitable for dyeing, at least one sulphur compound chosen from compounds of formula (II) and of formula (III):
R—SH (II)
R—S—S—R′ (III)
wherein R and R′, which may be identical or different, are chosen from saturated and unsaturated, branched and unbranched groups comprising from 1 to 100 carbon atoms, optionally comprising from 1 to 30 unsaturations, and which may comprise at least one hetero atom chosen from nitrogen, sulphur, oxygen and phosphorus, R and R′ possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, amino, C₁-C₄ monoalkylamino, C₁-C₄ dialkylamino, C₁-C₄ monohydroxyalkylamino, C₁-C₄ dihydroxyalkylamino, nitro, sulphonato, ammonio, C₁-C₄ trialkylammonio, imidazolio, pyridinio and benzothiazolio radicals.

37. The device according to claim 36, wherein composition (b) further comprises at least one amino acid.

38. The device according to claim 37, wherein the at least one amino acid is chosen from glycine, alanine, valine, leucine, isoleucine, methionine, phenylalanine, proline, serine, threonine, cysteine, asparagine, glutamine, tyrosine, histidine, lysine, ornithine, arginine, aspartic acid, glutamic acid and tryptophan.

39. The device according to claim 36, wherein at least one of compositions (a) and (b) further comprise at least one compound chosen from surfactants, polyols, polyol ethers, and aromatic monoalcohols.

40. The device according to claim 36, further comprising a third compartment comprising a composition (c) comprising, in a medium suitable for dyeing, at least one amino acid.

41. The device according to claim 40, wherein at least one of compositions (a), (b) and (c) further comprises at least one compound chosen from surfactants, polyols, polyol ethers, and aromatic monoalcohols.

42. A process for the optical lightening of keratin fibers, comprising applying to keratin fibers for a leave-in time sufficient to optically lighten the keratin fibers at least one composition comprising, in a medium suitable for dyeing:
at least one compound chosen from ortho- and α-dialdehyde compounds of formula (I): 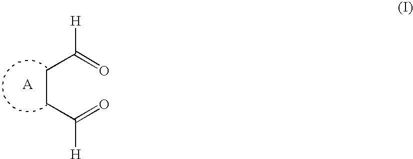
wherein A is chosen from:
fused and non-fused, aromatic and non-aromatic monocarbocyclic and polycarbocyclic groups comprising from 6 to 50 carbon atoms;
fused and non-fused, aromatic and non-aromatic, 5- to 30-membered monoheterocyclic and polyheterocyclic groups comprising at least one hetero atom chosen from nitrogen, sulphur, oxygen and phosphorus;
the group A possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, nitro, sulphonato, ammonio, C₁-C₄ trialkylammonio, imidazolio, pyridinio and benzothiazolio radicals; and
at least one sulphur compound chosen from compounds of formula (II) and of formula (III):
R—SH (II)
R—S—S—R′ (III)
wherein R and R′, which may be identical or different, are chosen from saturated and unsaturated, branched and unbranched groups comprising from 1 to 100 carbon atoms, optionally comprising from 1 to 30 unsaturations, and which may comprise at least one hetero atom chosen from nitrogen, sulphur, oxygen and phosphorus, R and R′ possibly being substituted with at least one radical chosen from halo, C₁-C₄ alkyl, hydroxyl, C₁-C₄ alkoxy, hydrogenocarbonyl, C₁-C₄ alkylcarbonyl, C₁-C₄ alkylthio, C₁-C₄ alkylcarboxyl, amino, C₁-C₄ monoalkylamino, C₁-C₄ dialkylamino, C₁-C₄ monohydroxyalkylamino, C₁-C₄ dihydroxyalkylamino, nitro, sulphonato, ammonio, C₁-C₄ trialkylammonio, imidazolio, pyridinio and benzothiazolio radicals.

43. The process according to claim 42, wherein the at least one composition further comprises at least one amino acid.

44. The process according to claim 42, wherein the at least one composition further comprises at least one compound chosen from surfactants, polyols, polyol ethers, and aromatic monoalcohols.
